(19) United States
(12) Patent Application Publication
(10) Pub. No.: US 2025/0235277 A1
(43) Pub. Date: Jul. 24, 2025

(54) ULTRA-MANEUVERABLE SURGICAL MICRO-ROBOT AND METHOD OF USE

(71) Applicant: Mordechai AVISAR, Highland Heights, OH (US)

(72) Inventor: Mordechai AVISAR, Highland Heights, OH (US)

(21) Appl. No.: 19/035,456

(22) Filed: Jan. 23, 2025

Related U.S. Application Data
(60) Provisional application No. 63/624,064, filed on Jan. 23, 2024.

(51) Int. Cl. A61B 34/37; A61B 5/00; A61B 5/055; A61B 34/00; A61B 34/30; A61M 25/01

(52) U.S. Cl. CPC A61B 34/37; A61B 5/0036; A61B 5/055; A61B 34/74; A61B 2034/301; A61B 2034/302; A61B 2034/731; A61M 25/0127

(57) ABSTRACT

A robotic medical device system for performing an operation on a patient includes a magnetic field source configured to generate a first magnetic field, an electrical current source configured to generate one or more selective electrical currents, and a micro robotic arm inserted within the patient. The micro robotic arm includes semi-flexible tubing configured to house a tool for performing the operation within the patient, and one or more joints formed in the semi-flexible tubing. Each of the joints includes a magnetic coil that is wrapped around the semi-flexible tubing. The magnetic coil is configured to receive one of the selective electrical currents generated by the electrical current source and generate a second magnetic field from the received one of the selective electrical currents. Each joint is configured to move the tool within the patient in accordance with an interaction between the first magnetic field and the second magnetic field.

### CROSS-REFERENCE TO RELATED APPLICATIONS

This application claims the benefit of U.S. Provisional Patent Application No. 63/624,064, filed Jan. 23, 2024, which is incorporated by reference herein in its entirety.

### FIELD OF THE INVENTION

This application relates generally to robotic medical device systems, and, more specifically, an ultra-maneuverable surgical micro-robot used to perform minimally invasive surgical operations with reduced local trauma.

### BACKGROUND OF THE INVENTION

Conventional approaches for performing surgery include creating incisions in the body and performing exploratory investigation in the body, both of which can do damage to the body in question.

For example, with respect to procedures involving the brain, most brain pathologies must be accessed through a Craniotomy, which includes drilling and removing part of the skull bone. The Craniotomy must be large enough to allow for sufficient access, maneuverability, and visibility to treat the pathology. Through the Craniotomy, surgeons navigate tools around the brain's anatomy toward the pathology, often needing to push various structures of the brain aside to access the pathology. This process compresses healthy brain tissue, thereby resulting in what is commonly referred to as “local trauma”. Further, sometime the size of a Craniotomy rivals that of a coffee cup.

It takes six to eight weeks for the Craniotomy wound to heal, during which no subsequent treatment pertinent to the pathology, such as radiation or chemotherapy, can be performed. The drawback of this is readily apparent, as aggressive tumors such as glioblastoma can grow as fast as fifteen percent per day. As such, the Craniotomy process directly diminishes the margins of hospitals involved in performing them, as it results in provision of suboptimal treatment.

Robotic approaches to surgery have been developed that reduce the impact on the patient and improve the chances of success, but there are shortcomings to current devices and surgical techniques. The lack of maneuverability of robotic surgical instruments has limited the utility of such devices, which has interfered with the most desirable outcomes in patient surgery.

Currently, there are approaches to robotic surgery that utilize the electromagnetic fields used to perform magnetic resonance imaging (MRI) to provide motive power to robotic devices used in surgical situations. These approaches are built on Laser ablation, RF ablation, drug delivery, chemotherapy delivery, and combination chemotherapy heated delivery methodologies, along with robotic surgery techniques. However, the devices utilized in these approaches have limited maneuverability and are not sufficient for use in more complicated surgeries.

FIGS. 1-3 illustrate an example of a conventional tube/catheter robotic arm 2 inserted in a pathology 4. These drawings also illustrate a stepwise laser ablation example in which the construction of the conventional tube/catheter robotic arm 2 requires treatment of a brain 6 along a linear manner, thereby making it possible for sections of pathology 4 to be missed. As is illustrated in FIGS. 1 and 2, the conventional tube/catheter robotic arm 2 illustrated therein is unable to curve or maneuver in non-linear directions, necessitating a straight ablation pattern for the conventional tube/catheter robotic arm 2. FIGS. 2 and 3 illustrate the effects of the straight laser ablation treatment that results in treated areas 8 of the pathology 4 and untreated areas 10 of the pathology 4. Multiple operations or at least multiple penetrations may be needed to treat the initially untreated areas 10 of the pathology 4, thereby resulting in increased time and effort on the part of the surgeon and simultaneously increasing risk to the brain 2.

The present invention has been developed to address these and other issues by providing a robotic medical device that includes a micro robotic arm capable of delicate maneuvering in the human body, thereby enabling surgical procedures to be performed without major surgical incisions or intrusions, as would be required with the use of catheters, probes, and endoscopes. The present invention also provides a device that is able utilize the electromagnetic energy of a scanning (imaging) machine, such as an MRI device, to power the motive functions of the micro robotic arm, thereby guiding it through portions of the patient's body.

### SUMMARY OF THE INVENTION

In accordance with an embodiment of the present invention, there is provided a robotic medical device system for performing an operation on a patient. The system includes a magnetic field source configured to generate a first magnetic field, an electrical current source configured to generate one or more selective electrical currents, and a micro robotic arm inserted within the patient. The micro robotic arm includes semi-flexible tubing configured to house a tool for performing the operation within the patient, and one or more joints formed in the semi-flexible tubing. Each of the joints includes a magnetic coil that is wrapped around the semi-flexible tubing. The magnetic coil is configured to receive one of the selective electrical currents generated by the electrical current source and generate a second magnetic field from the received one of the selective electrical currents. Each of the joints is configured to move the tool within the patient in accordance with an interaction between the first magnetic field and the second magnetic field.

In accordance with yet another embodiment of the present invention, there is provided a method of performing an operation on a patient with a robotic medical device system. A magnetic field is generated near the patient using a magnetic field source. A micro robotic arm is inserted within the patient. The micro robotic arm includes semi-flexible tubing and one or more joints formed in the semi-flexible tubing. The semi-flexible tubing is configured to house a tool for performing the operation within the patient. Each of the joints include a magnetic coil that is wrapped around the semi-flexible tubing. The magnetic coil is configured to receive one of one or more selective electrical currents generated by an electrical current source and generate a second magnetic field from the received one of the selective electrical currents. The tool is moved within the patient in accordance with an interaction between the first magnetic field and the second magnetic field.

### BRIEF DESCRIPTION OF THE DRAWINGS

The features and advantages of the example embodiments described herein will become apparent to those skilled in the art to which this disclosure relates upon reading the following description, with reference to the accompanying drawings, in which:

FIG. 1 is a schematic view illustrating an example of a conventional tube/catheter robotic arm preparing to perform an operation on a pathology of a brain;

FIG. 2 is a schematic view illustrating an example of the conventional tube/catheter robotic arm performing an operation on the pathology of the brain illustrated in FIG. 1 in a linear manner;

FIG. 3 is a schematic view illustrating an example showing the results of the conventional tube/catheter robotic arm performing an operation on the pathology of the brain illustrated in FIGS. 1 and 2;

FIG. 4 is a schematic view illustrating an example of a robotic medical device system in which a micro robotic arm of the present invention is implemented;

FIG. 5 is a schematic view illustrating an example of the micro robotic arm of the present invention mounted near a patient's head;

FIG. 6 is a schematic view illustrating an example of the micro robotic arm of the present invention preparing to perform an operation on a pathology of a brain;

FIG. 7 is a schematic view illustrating another example of the micro robotic arm of the present invention preparing to perform an operation on the pathology of the brain illustrated in FIG. 6;

FIG. 8 is a schematic view illustrating an example of the micro robotic arm of the present invention beginning to perform an operation on the pathology of the brain illustrated in FIG. 6;

FIG. 9 is a schematic view illustrating an example of the micro robotic arm of the present invention continuing to perform the operation on the pathology of the brain initiated in FIG. 8;

FIG. 10 is a schematic view illustrating an example of the micro robotic arm of the present invention continuing to perform the operation on the pathology of the brain continued in FIG. 9;

FIG. 11 is a schematic view illustrating an example of the micro robotic arm of the present invention completing the operation on the pathology of the brain illustrated in FIG. 7; and

FIG. 12 is a schematic view illustrating an alternate example of the micro robotic arm of the present invention completing the operation on the pathology of the brain illustrated in FIG. 7.

### DETAILED DESCRIPTION OF THE INVENTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. In addition, descriptions of functions and constructions that are well known to one of ordinary skill in the art may be omitted for increased clarity and conciseness.

Throughout the drawings and the detailed description, the same reference numerals refer to the same elements. The drawings may not be to scale, and the relative size, proportions, and depiction of elements in the drawings may be exaggerated for clarity, illustration, and convenience.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

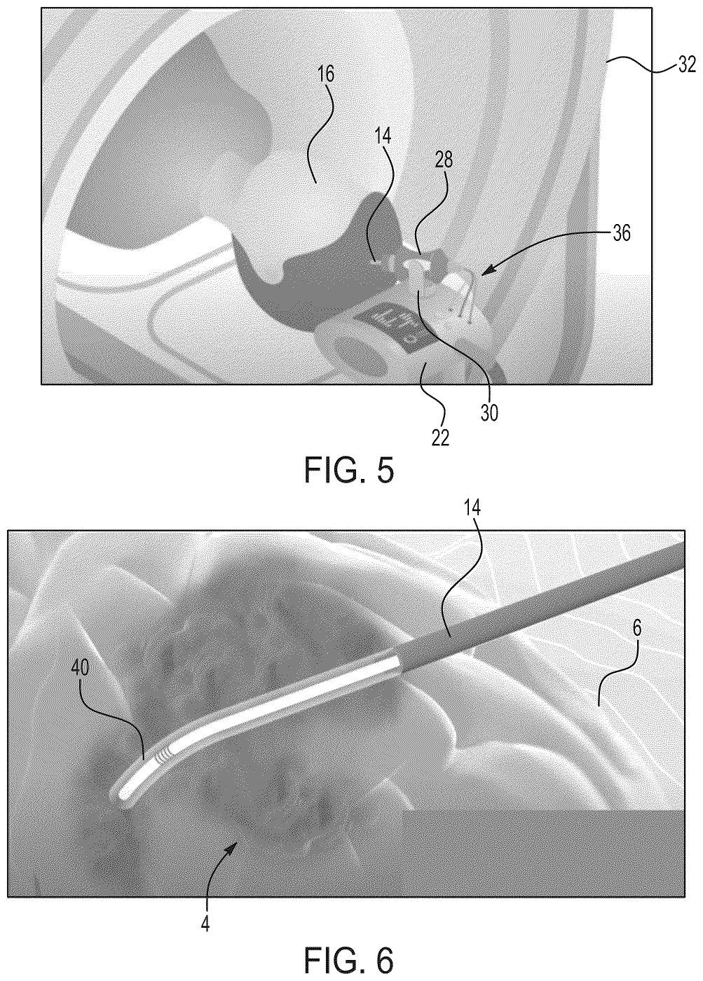

FIG. 4 is a schematic view illustrating an example of a robotic medical device system 12 in which a micro robotic arm 14 of the present invention is implemented. FIG. 5 is a schematic view illustrating an example of the micro robotic arm 14 of the present invention mounted by a patient's head 16. FIGS. 6-11 are schematic views illustrating examples of the micro robotic arm 14 of the present invention performing an operation on a pathology 4 of the brain 6. FIG. 12 is a schematic view illustrating another example of the micro robotic arm 14 of the present invention performing an operation on a pathology 4 of the brain 6.

The robotic medical device system 12 and the micro robotic arm 14 may be used to perform different surgical procedures in hospital environments. As is illustrated herein, one example of such a procedure includes management of laser ablation of a pathology 4 in the brain 6. However, embodiments disclosed herein are not limited thereto. For example, it is contemplated that the micro robotic arm 14 and at least a substantial portion of the system 12 could be implemented in any environment in which solutions are desired for robotic devices to perform accurate and minimally invasive procedures using minute movements, such as, but not limited to, targeted drug delivery of precision medicine, semiconductor manufacturing, micropackage assembly, and any procedure that could benefit from remote operation.

In the non-limiting example described herein, the system 12 includes a computing complex 18. The computing complex 18 may include one or more processors and one or more means of storage, but is not limited thereto. The processors and the storage of the computing complex 18 may be oriented, positioned, or connected in any way to facilitate proper operation of the computing complex 18. This includes, but is not limited to, wired configurations, wireless configurations, local configurations, wide area configurations, and any combination thereof in which communication therebetween can be established through compatible network protocol.

Other than what is specifically pointed out in the discussion below, much of the operation of the components illustrated in FIGS. 4 and 5 that make up the system 12 and the computing complex 18 and perform the method and operations described herein are implemented by hardware components. For example, one of the components contemplated for implementation in the present invention is a control joystick 20 that is connected to a control pad 22 positioned adjacent to the patient's head 16 and enables remote maneuvering of the micro robotic arm 14. The use of a mouse (not illustrated) and a keyboard 24 with the computing complex 18 as input devices along with the control joystick 20 to control the computing complex 18 is also contemplated. At least one display device 26 is contemplated for use in the present invention for the display of real time MRI images to assist the performance of procedures and view the status of the computing complex 18.

Examples of hardware components are not limited to the above-described or yet-to-be described example apparatuses, units, modules, and devices and may include controllers, sensors, generators, drivers, and any other electronic components known to one of ordinary skill in the art. Such components may be variably located according to design needs and may communicate with each other through wired or wireless means.

The processors may be implemented by one or more processing elements. Such processing elements may be as an array of logic gates, a controller and an arithmetic logic unit, a digital signal processor, a microcomputer, a programmable logic controller, a field-programmable gate array, a programmable logic array, a microprocessor, or any other device or combination of devices known to one of ordinary skill in the art that is capable of responding to and executing instructions in a defined manner to achieve a desired result.

For simplicity, the singular term “processor” may be used in the description of the example processors described herein, but in other examples multiple processors are used, or the processor includes multiple processing elements, or multiple types of processing elements, or both. In one example, the system 12 includes multiple processors in the computing complex 18, and in another example, a hardware component of system 12 includes an independent processor or another controller containing a processor, which then communicates data to receive data from the processor of the computing complex 18. The processor of the computing complex 18 may be defined as a hardware component, along with other components of the system 12 discussed below. Similar to the processor and other hardware components containing processing functionality may be defined according to any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing. The processor may be connected via cable or wireless network to hardware components to provide instruction thereto or to other processors to enable multiprocessing capabilities.

Instructions or software to control the processor or hardware including processors within the system 12 to implement the hardware components and perform the methods as described below are written as computer programs, code segments, instructions or any combination thereof, for individually or collectively instructing or configuring the processor or hardware including processors within the system 12 to operate as a machine or special-purpose computer to perform the operations performed by the hardware components and the methods as described below. In one example, the instructions or software include machine code that is directly executed by the processor or hardware including processors within the system 20, such as machine code produced by a compiler. In another example, the instructions or software include higher-level code that is executed by a processor or hardware including processors within the system 12 using an interpreter.

Hardware components implemented in the system 12, such as a processor or components linked to the processor, execute instructions or software, such as an operating system (OS) and one or more software applications that run on the OS, to perform the operations described here below.

The instructions or software to control a processor or hardware including processors within the system 12 to implement the hardware components and perform the methods as described below, and any associated data, data files, and data structures, are recorded, stored, or fixed in storage. Storage of the computing complex 18 generically refers to one or more memories storing instructions or software that are executed by a processor. However, the hardware components implemented in the system 12, such as a processor or components linked to a processor, may include local storage or access, manipulate, process, create, and store data in a storage in response to execution of the instructions or software.

Storage may be represented by one or more non-transitory computer-readable storage media. Storage may be representative of multiple non-transitory computer-readable storage media linked together via a network of the computing complex 18. For example, non-transitory computer-readable storage media may be located in one or more storage facilities or one or more data centers positioned remotely from the system 12 within the computing complex 18. Such media may be connected to the system 12 through a network of the computing complex 18. The network of the computing complex 18 allows the non-transitory computer-readable storage media remotely located at the data center or the storage facility to transfer data over the network to non-transitory computer-readable storage medium within storage of the computing complex 18. In addition, storage may be representative of both remotely and locally positioned non-transitory computer-readable storage media.

Examples of a non-transitory computer-readable storage medium include read-only memory (ROM), random-access memory (RAM), flash memory, solid state memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor of the computing complex 18 or hardware including processors within the system 12 so that a processor or processors can execute the instructions. In one example, the instructions or software and any associated data, data files, and data structures are distributed over network-coupled computer systems so that the instructions and software and any associated data, data files, and data structures are stored, accessed, and executed in a distributed fashion by a processor.

The micro robotic arm 14 of the present invention is constructed from semi-flexible tubing having a diameter between 2 mm and 4 mm, which is sufficiently sized to house a tool, such as a laser ablation tool, a micro-biopsy needle, a micro-syringe for drug and chemotherapy delivery, a micro-camera for enhanced visualization, and a micro-bipolar device for precision cauterization. The micro robotic arm 14 can also serve as the access port for multiple instruments, such as a laser ablation tool, a camera, a bipolar tool, a sensor, another type of laser tool, or an RF ablation tool.

The micro robotic arm 14 is mounted in a base 28. The base 28 securely holds the micro robotic arm 14 for implementation in a procedure. The base 28 is mounted on the control pad 22 by a control pad mount 30 that is provided on the control pad 22. The control pad mount 30 fixes the base 28 in a position that allows the micro robotic arm 14 to fulfill its intended purpose. In the instant case, the micro robotic arm 14 is being used to perform procedures and operations concerning the brain 6 within the patient's head 16.

It is noted that the patient whose head 16 is under operation is positioned within an MRI device 32. The MRI device 32 can be used to provide real time imagery to the display devices 26 to assist an operator 34 in completion of the procedure being performed by the micro robotic arm 14. In the instant case, the MRI device 32 can also be used to enable control of the micro robotic arm 14, as will be further described below.

The base 28 connects the micro robotic arm 14 to a plurality of wires 36 extending from and through the control pad 22. In the present case, some of the wires 36 are associated with control of the micro robotic arm 14. In addition, since the micro robotic arm 14 in this example houses a tool being used to perform laser ablation on a pathology 4 in the brain 6 of a patient's head 16, some of the wires 36 provide fiber optic cooling to the micro robotic arm 14 for the laser ablation that is administered by the micro robotic arm 14. It is noted that embodiments disclosed herein are not limited thereto, and the wires 36 may provide anything needed to the micro robotic arm 14 in view of how the micro robotic arm 14 is to be implemented.

At least some of the wires 36 are connected via a control conduit 38 to the computing complex 18. In one example, the control joystick 20, the mouse (not illustrated), the keyboard 24, or the computing complex 18 as a whole may provide either automated control instructions or control instructions specified by an operator 34 to the micro robotic arm 14 via the wires 36 and the control conduit 38. Input devices that can be used by the operator 34 are not limited to those described herein and could include virtual reality headsets or handsets. In addition, the wires 36 and the control conduit 38 may provide the computing complex 18 with feedback and imagery from the micro robotic arm 14 to be displayed on the display devices 26 of the computing complex 18 to the operator 34 depending on what purpose the micro robotic arm 14 is being used to achieve.

It is noted that FIG. 4 illustrates an example of the robotic medical device system 12 in which the operator 34 in charge of operating the micro robotic arm 14 is positioned at the computing complex 18, which is outside of the room in which the MRI device 32, the patient, and the micro robotic arm 14 are positioned. The displays 26 allow the operator 34 to remotely operate the micro robotic arm 14 from the input device previously mentioned with respect to the computing complex 18. However, while remote operation of the micro robotic arm 14 is a benefit, embodiments disclosed herein are not limited thereto. It is perceivable that the computing complex 18 can be placed in the same room in which the operation with the micro robotic arm 14 takes place, thereby requiring the operator 34 to be in the same room as the MRI device 32, the patient, and the micro robotic arm 14.

In the instant case, the micro robotic arm 14 has at least one joint 40. The joints 40 may include springs, semi-flexible elements, or other similar mechanisms that are supportive of controlled, precise motion. One or more magnetic coils are embedded in and wrapped around each of the joints 40, as illustrated in FIGS. 6-12. Each of the coils are connected to the wires 36 associated with control of the micro robotic arm 14. Each of the coils can receive a selective electrical current driven from the computing complex 18 through the control conduit 38 to the wires 36 associated with control of the micro robotic arm 14. Respectively driving electrical currents into the coils of the joints 40 results in generation of targeted and localized magnetic fields around the coils and subsequently the respective joints 40 around which the coils are wound.

The targeted magnetic fields magnetically interact with a strong, static magnetic field generated by the MRI device 32, thereby producing torque and forces in selected axes. The torque and forces result in the controlled curvature of the joints 40, along with controlled movement and rotation along one or more axes or in one or more directions. The curvature and movement of the joints 40 occurs due to the Lorentz force arising from the interaction between the current-carrying magnetic fields of the coils respectively associated with the joints 40 and the static magnetic field of the MRI device 32. The MRI device 32 provides the static magnetic environment necessary for the interactions, but the current in the coils respectively associated with the joints 40 generates the localized magnetic field and enables movement. The mechanical elements of the joints 40 work in tandem with the Lorentz force to stabilize and fine tune the movement of the micro robotic arm 14. As a result, the operation of the micro robotic arm 14 relies on the presence of the strong, static magnetic field of the MRI device 32 to enable the Lorentz force interactions with the magnetic fields produced by the coils of the joints 40.

The selective electrical currents sent from the computing complex 18 to the coils correspond with automated control instructions or control instructions specified by an operator 34 provided from the computing complex 18 through the keyboard 24, the control joystick 20, or the mouse (not illustrated) to the micro robotic arm 14. Moreover, the activating trigger for each joint 40 to be moved can be coded separately or be sent to all the joints 40 at the same time by the computing complex 18 through the control conduit 38. In addition, the magnetic field activation of the coils allows each one of the joints 40 to be moved with respect to multiple axes and rotation about those axes (for example, six degrees of freedom, including X-axis, Y-axis, Z-axis, Roll, Pitch, and Yaw). Every axis in every micro robotic arm 14 can be controlled separately. Further, the stiffness of each joint 40 can be independently specified to be stiffer than normal or softer than normal. These interactions enable the micro robotic arm 14 to be snaked through anatomic structures on the way to reaching its target pathology 4 while avoiding eloquent structures within the brain 6 along the way.

According to the instructions provided to the coils of the joints 40, the magnetic fields generated by the MRI device 32 can result in the curving or the maneuvering of one joint 40 separately from the curving or the maneuvering of another joint 40, thereby achieving a snake like maneuverability of the micro robotic arm 14 and enabling a trajectory through the patient's body or organ to reach a broader and more difficult to reach locations. The environment of the MRI device 32 is supportive for performing comprehensive surgical tasks with one of the previously referenced tools housed within or supported by the micro robotic arm 14. In this way, the micro robotic arm 14 can be used to support and treat many different investigative and/or surgical procedures in a highly flexible and maneuverable manner.

The design of the joints 40 accommodate the forces generated by the interaction of the magnetic field generated by the MRI device 32 and the magnetic fields of the coils of the joints 40. Depending on the design, the joints 40 can either constrain motion to a specific path or allow dynamic positioning. The magnetic fields of the MRI device 32 and the coils of the joints 40 control the movement, while the joints 40 ensure the movement occurs within the desired parameters.

In one example, one or more of the joints 40 are spring-loaded, thereby incorporating a mechanical spring that provides elasticity and controlled resistance. The spring-loaded joints can store elastic energy and use it to stabilize movements caused by Lorentz force, thereby preventing excess force or vibrations from causing unintended damage to surrounding tissue. This allows for smooth bending or rotation under the influence of the Lorentz force, making the spring-loaded joints suitable for the micro-manipulations required in navigating soft tissues in surgery of the brain 6. The spring-loaded joints naturally return to their original position when Lorentz force is removed.

In another example, one or more of the joints 40 are single-axis pivot joints, allowing rotation or movement in three dimensions about a fixed axis and acting like a hinge, enabling the micro robotic arm 14 to bend or rotate in a single plane (e.g., forward/backward or up/down). Single-axis pivot joints are useful for tasks requiring controlled movement along a single axis, such as insertion of surgical tools at fixed angles.

In an additional example, one or more of the joints 40 are three-dimensional (3D) multi-axis joints, allowing rotations or movement in three directions. The 3D multi-axis joints often use a ball-and-socket configuration or interconnected pivots to achieve flexibility in all directions. The 3D joints can maximize the flexibility needed to position the micro robotic arm 14 in complex orientation. These types of joints are critical for navigating irregular geometries, such as reaching a specific point in a curved or multi-layered structure of the brain 6. These types of joints also act as a safeguard by absorbing excess force or vibrations caused by Lorentz force, thereby preventing unintended damage to surrounding tissue.

In yet another example, one or more of the joints 40 are hybrid joints, combining a spring mechanism with multi-axis flexibility. The spring ensures stability and self-centering, while the multi-axis design provides freedom of movement in various directions. Hybrid joints are particularly useful in surgical applications where maintaining alignment while allowing for free movement is crucial.

In still another example, one or more of the joints 40 are flexible joints with semi-rigid materials, such as polymers or flexible metals, that bend under force but retain enough stiffness to return to an original shape. Such joints are flexible in applications where minimizing weight and size is a priority, such as navigating fine structures in the brain 6.

In an example application of the joints 40 in a surgery of the brain 6 targeting Glioblastoma Multiforme (GBM), the micro robotic arm 14 includes a number of different types of joints 40. Single-axis pivot joints guide a tool housed by the micro robotic arm 14 into the target area of the pathology 4 at a precise angle. 3D multi-axis joints navigate around critical structures, such as blood vessels or nerves. Further, spring-loaded joints ensure safe and controlled retraction or positioning of the tool during the procedure.

While the example illustrated herein implements the use of magnetic fields to control the curvature and maneuvering of the micro robotic arm 14, embodiments disclosed herein are not limited thereto. The joints 40 of the micro robotic arm 14 can also be controlled using frequency, I modulated control, or digital code control. Further, while the MRI device 32 is implemented to provide magnetic fields to interact with the magnetic fields generated by the electrical current supplied to the coils of the joints 40 of the micro robotic arm 14, it is contemplated that any device capable of providing strong, static magnetic fields could enable maneuverability of the joints 40 of the micro robotic arm 14 when electrical current is supplied to the coils of the joints 40 of the micro robotic arm 14 to produce the localized magnetic fields.

The operation of the control of micro robotic arm 14 and the joints 40 can be automated by use of an algorithm with the computing complex 18, manually controlled by the operator 34 using the keyboard 24, the control joystick 20, and the mouse (not illustrated), or a combination of both. The computing complex 18 can take input regarding the entry point of the micro robotic arm 14, the target of the micro robotic arm 14, and all the locations within the target to be treated to enable an algorithm to control the micro robotic arm 14 using the MRI device 32. In this case, specific areas of the brain 6 in the patient's head 16 serve as the target of the micro robotic arm 14. The algorithm takes into consideration the anatomical structures between the entry point and the target, the size and shape of the pathology 4, and the obstacles or anatomical structures positioned adjacent to or blocking the target and the pathology 4. This is done to preserve, and subsequently avoid damage to, untargeted portions of the brain 6. Historical consideration of outcome and issues can also be programmed into the algorithm to promote the best path for micro robotic arm 14 to apply treatment.

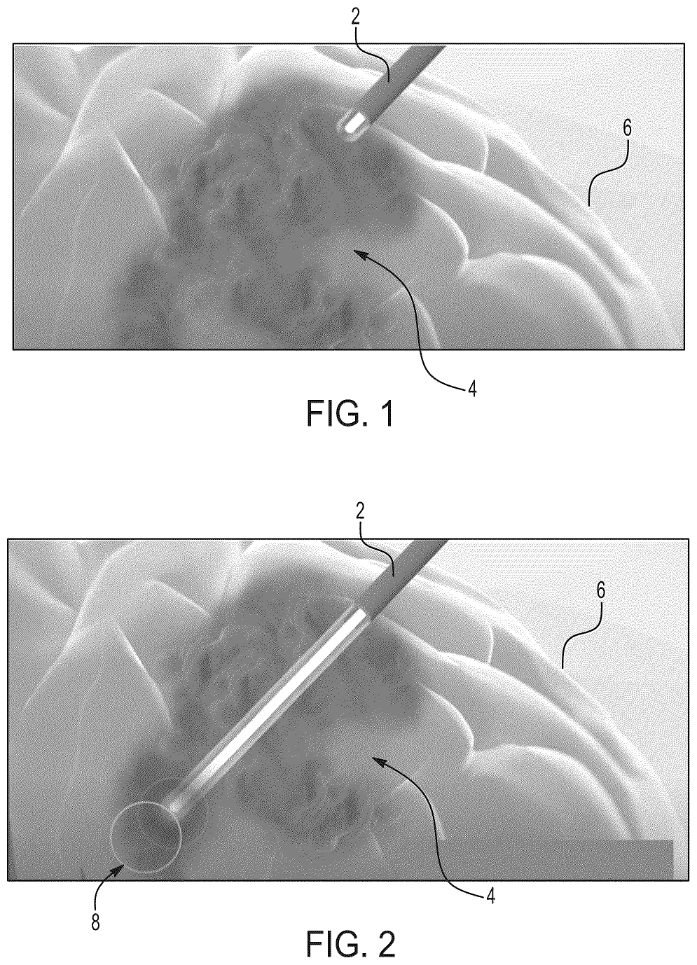

FIGS. 6-11 illustrate an example of the micro robotic arm 14 of the present invention performing an operation on the pathology 4 of the brain 6. These drawings also illustrate a stepwise laser ablation example, like that which is shown in FIGS. 1-3 with respect to the convention tube/catheter robotic arm 2.

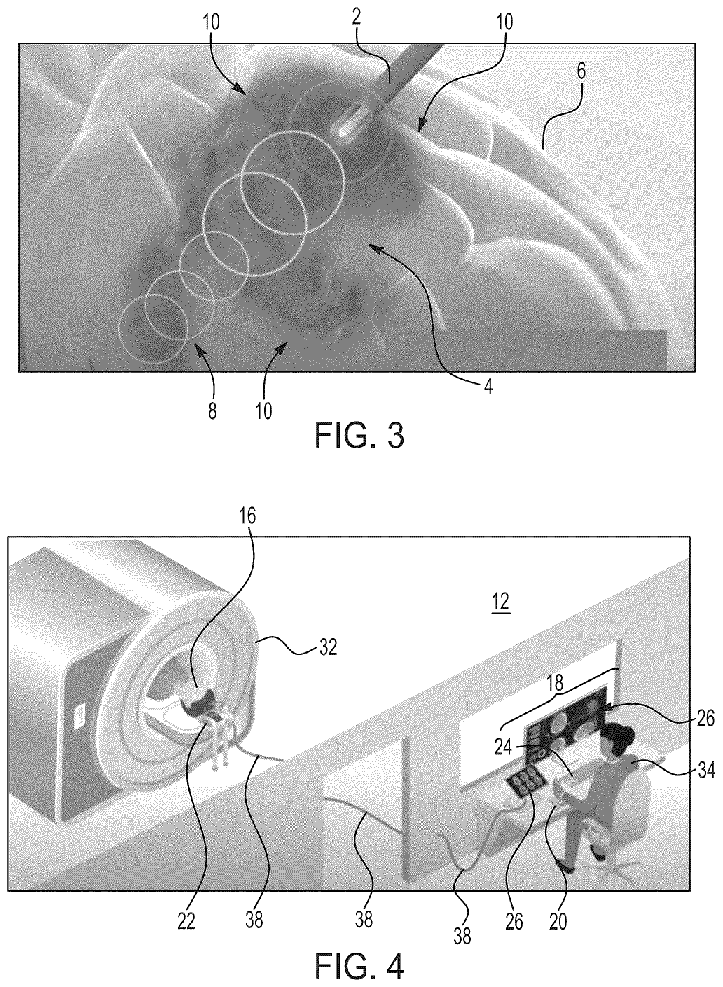

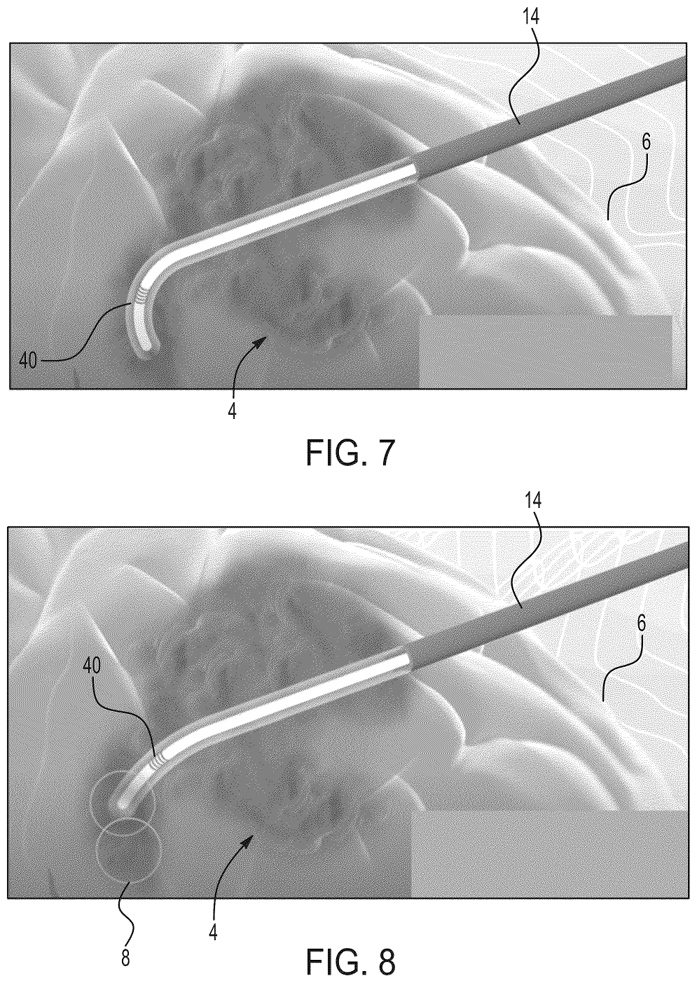

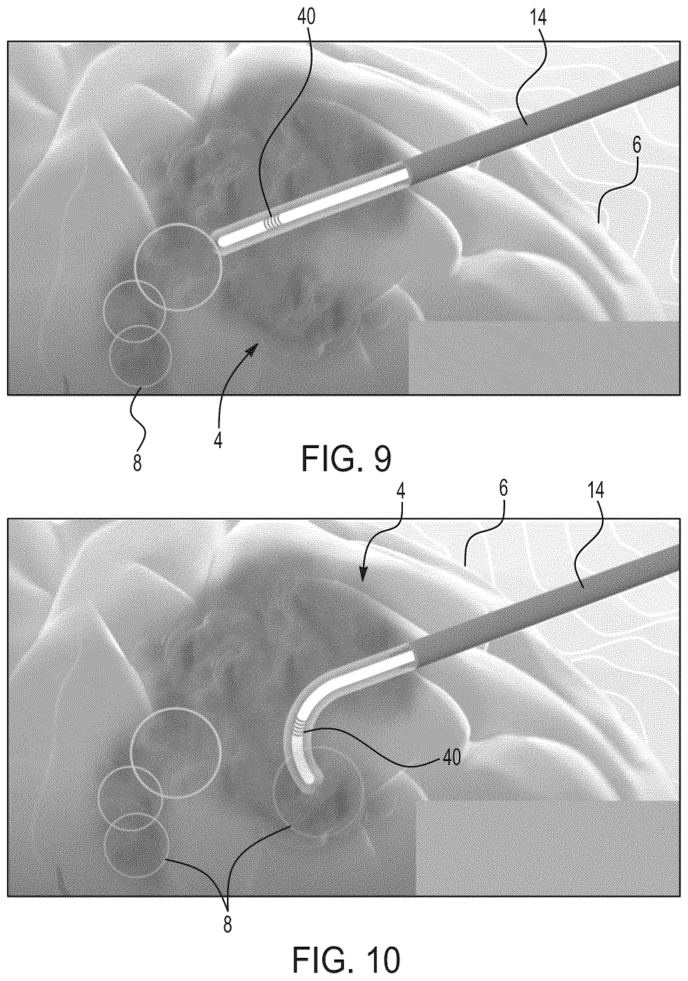

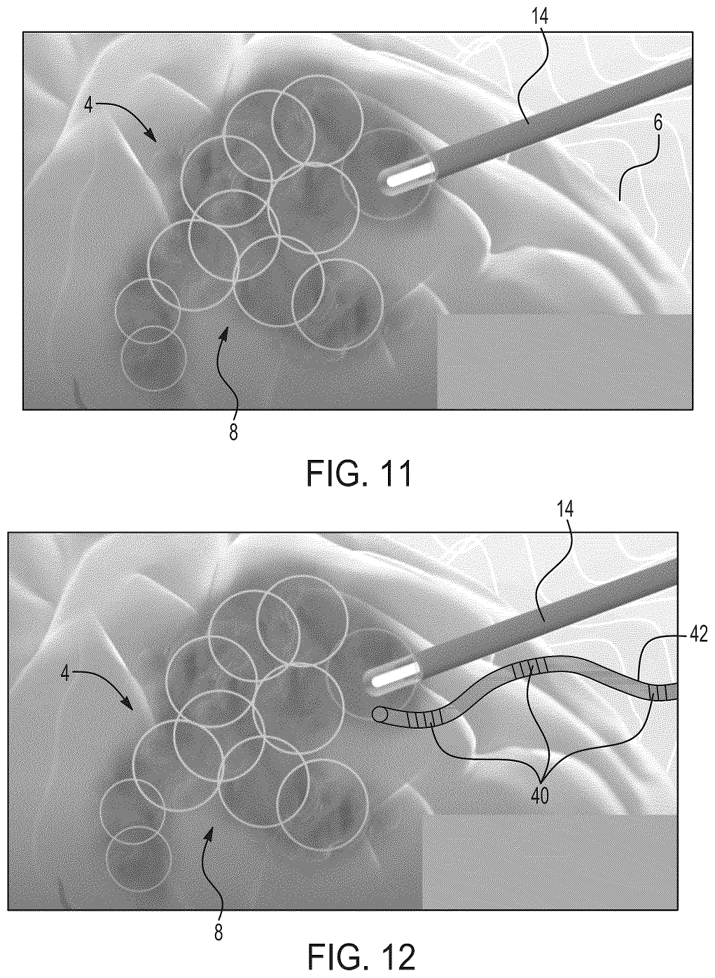

Illustrated in FIGS. 6 and 7, magnetic fields generated by the MRI device 32 have resulted in a curvature of the joint 40 according to instructions, i.e., electrical current, received by the surrounding coils in preparation for laser ablation of portions of the pathology 4. FIG. 8 illustrates areas 8 of the pathology 4 that are treated via laser ablation using the micro robotic arm 14 with a laser ablation tool. In FIGS. 9 and 10, the curvature of the joint 40 has been adjusted, thereby indicating that the electrical current received by the coils surrounding the joint 40 has changed in such a way to generate a magnetic field allowing the magnetic fields generated by the MRI device 32 to curve the joint 40 of the micro robotic arm 14 in another direction in preparation for treating other areas of the pathology 4. FIG. 10 specifically illustrates the treatment of an area 8 of the pathology 4 by the micro robotic arm 14 that was an untreated area 10 and not able to be treated by the conventional tube/catheter robotic arm 2 as illustrated in FIG. 3 without multiple penetrations of the convention tube/catheter robotic arm 2 or the reinsertion of the conventional tube/catheter robotic arm 2 at another angle. As is illustrated in FIGS. 9 and 10, such a reinsertion is not required with the use of the micro robotic arm 14. Finally, FIG. 11 illustrates the treatment of all areas 8 of the pathology 4 without removal of the micro robotic arm 14 from the theater of the brain 6.

As a result of the micro robotic arm 14 to operate on a curve, the entire pathology 4 can be treated in one operation with only one penetration. This is in contrast with the conventional tube/catheter robotic arm 2, which is limited to linear operation and may require multiple penetrations to properly treat the patient, if such treatment is even possible. Arthroscopic surgery can be performed in an accurate and less invasive manner using the micro robotic arm 14. The micro robotic arm 14 curves and maneuvers in minute and accurate movements through the body to avoid critical organs and vessels and reach regions for surgical treatment that can be missed using traditional treatments. Accuracies of less than 2 mm are possible.

FIG. 12 illustrates another example of a micro robotic arm 42. The micro robotic arm 42 illustrated in FIG. 12 includes multiple joints 40 surrounded by magnetic coils. This allows additional maneuvering capabilities. Further, while the micro robotic arm 14 is illustrated as performing a laser ablation with a laser ablation tool, the micro robotic arm 42 does not have to duplicate the purpose of the micro robotic arm 14. The micro robotic arm 42 can be implemented as any desired device to observe and/or treat the pathology 4, such as a camera, a bipolar tool, another type of laser tool, an ultrasound sensor, a heat sensor, or an RF ablation tool. In this way, the micro robotic arm 42 can be used to support and treat many different investigative and/or surgical procedures in a highly flexible and maneuverable manner at the same time the micro robotic arm 14 is serving its own specific purpose. In other words, multiple micro robotic arms can be used in the same operation theater for differing and complimentary purposes. Moreover, the operation of the micro robotic arm 14 may be manual by the operator 34, while the operation of micro robotic arm 42 may be automated through a program stored in the computing complex 18, or vice versa.

While the computing complex 18 instructs the activation and control of movement of the joints 40 through the provision of selective electrical current to the corresponding coils via the control conduit 38 and the wires 36 connected to the coils, embodiments described herein are not limited thereto. For example, the movement of the joints 40 could be activated and controlled by any module configured to provide instructions via selective electrical current to the coils using the wires 36, including, but not limited to, the control pad 22.

It is also noted that the computing complex 18 or the control pad 22 may be configured to control operation of the tool housed by the micro robotic arm 14. For example, a laser ablation tool housed in the micro robotic arm 14 may be operated by instructions provided via a connection between the tool and the computing complex 18 or the control pad 22. Wires 36 may be used to communicate the instructions from the computing complex 18 or the control pad 22 to the tool housed in the micro robotic arm 14. Moreover, data transmitted from a tool, such as video images from a camera for display on the display devices 26, can be received by the computing complex 18 or the control pad 22 through the wires 36.

The foregoing description is a specific embodiment of the present invention. It should be appreciated that this embodiment is described for purposes of illustration only, and that numerous alterations and modifications may be practiced by those skilled in the art without departing from the spirit and scope of the invention. It is intended that all such modifications and alterations be included insofar as they come within the scope of the invention as claimed or the equivalents thereof.

What is claimed is:

1. A robotic medical device system for performing an operation within a patient, the system comprising:
a magnetic field source configured to generate a first magnetic field near the patient;
an electrical current source configured to generate one or more selective electrical currents; and
a micro robotic arm inserted within the patient, comprising:
semi-flexible tubing configured to house a tool for performing the operation within the patient; and
one or more joints formed in the semi-flexible tubing, each of the joints comprising a magnetic coil that is wrapped around the semi-flexible tubing, the magnetic coil being configured to receive one of the selective electrical currents generated by the electrical current source and generate a second magnetic field from the received one of the selective electrical currents, each of the joints being configured to move the tool within the patient in accordance with an interaction between the first magnetic field and the second magnetic field.

2. The system according to claim 1, wherein the patient is positioned within a magnetic resonance imaging (MRI) device, and
wherein magnetic field source is the MRI device.

3. The system according to claim 1, wherein the micro robotic arm is inserted into a brain of the patient, and
wherein the tool is configured to treat a pathology of the brain.

4. The system according to claim 1, wherein the electrical current source is a computing complex,
wherein the computing complex is connected to the magnetic coil of each of the joints, and
wherein the selective electrical currents correspond with instructions to move the tool processed by the computing complex.

5. The system according to claim 4, wherein the instructions to move the tool are communicated to the computing complex by a control joystick, a mouse, a keyboard, or a combination thereof operated by an operator.

6. The system according to claim 4, wherein the instructions to move the tool are communicated to the computing complex by data located on a non-transitory computer-readable storage medium and selected for execution by an operator.

7. The system according to claim 1, wherein each of the joints are configured to move in six degrees of freedom.

8. The system according to claim 1, wherein the joints are selected from a group consisting of spring-loaded joints, single-axis pivot joints, three-dimensional multi-axis joints, hybrid joints, and flexible joints with semi-rigid materials.

9. A method of performing an operation within a patient, comprising:
generating, using a magnetic field source, a first magnetic field near the patient;
inserting a micro robotic arm within the patient, the micro robotic arm including semi-flexible tubing and one or more joints formed in the semi-flexible tubing, the semi-flexible tubing being configured to house a tool for performing the operation within the patient, each of the joints including a magnetic coil that is wrapped around the semi-flexible tubing, the magnetic coil being configured to receive one of one or more selective electrical currents generated by an electrical current source and generate a second magnetic field from the received one of the selective electrical currents; and
moving the tool within the patient in accordance with an interaction between the first magnetic field and the second magnetic field.

10. The method according to claim 9, further comprising:
positioning the patient within a magnetic resonance imaging (MRI) device prior to the inserting of the micro robotic arm within the patient,
wherein the magnetic field source is the MRI device.

11. The method according to claim 9, wherein the micro robotic arm is inserted into a brain of the patient, and
wherein the tool is configured to treat a pathology of the brain.

12. The method according to claim 9, wherein the electrical current source is a computing complex,
wherein the computing complex is connected to the magnetic coil of each of the joints, and
wherein the selective electrical currents correspond with instructions to move the tool processed by the computing complex.

13. The method according to claim 12, wherein the instructions to move the tool are communicated to the computing complex by a control joystick, a mouse, a keyboard, or a combination thereof operated by an operator.

14. The method according to claim 12, wherein the instructions to move the tool are communicated to the computing complex by data located on a non-transitory computer-readable storage medium and selected for execution by an operator.

15. The method according to claim 9, wherein each of the joints are configured to move in six degrees of freedom.

16. The method according to claim 9, wherein the joints are selected from a group consisting of spring-loaded joints, single-axis pivot joints, three-dimensional multi-axis joints, hybrid joints, and flexible joints with semi-rigid materials.
